(19) United States
(12) Patent Application Publication
(10) Pub. No.: US 2024/0225860 A1
(43) Pub. Date: Jul. 11, 2024

(54) AMPLITUDE MODULATED HIGH-FREQUENCY STIMULATION TO ELICIT STOCHASTIC NEURAL SPIKING ACTIVITY

(71) Applicants: University of Pittsburgh - Of the Commonwealth System of Higher Education, Pittsburgh, PA (US); The Regents of the University of Michigan, Ann Arbor, MI (US)

(72) Inventors: Beatrice Barra, New York, NY (US); Lee Erik Bartholomew Fisher, Pittsburgh, PA (US); Marco Capogrosso, Pittsburgh, PA (US); Ehsan Mirzakhalili, Ann Arbor, MI (US); Scott Lempka, Ann Arbor, MI (US)

(73) Assignees: University of Pittsburgh - Of the Commonwealth System of Higher Education, Pittsburgh, PA (US); The Regents of the University of Michigan, Ann Arbor, MI (US)

(21) Appl. No.: 18/289,301

(22) PCT Filed: May 3, 2022
(86) PCT No.: PCT/US2022/027498

§ 371 Date: Nov. 2, 2023

Related U.S. Application Data
(60) Provisional application No. 63/183,552, filed on May 3, 2021.

(51) Int. Cl. A61F 2/70; A61N 1/36; A61F 2/50

(52) U.S. Cl. CPC A61F 2/70; A61N 1/36003; A61N 1/36071; A61N 1/361; A61N 1/36139; A61N 1/36157; A61N 1/36171; A61N 1/36192; A61F 2002/5064

(57) ABSTRACT

Methods are disclosed for stimulating asynchronous and stochastic neural activity in a subject in need thereof. In some embodiments, the methods are used to provide asynchronous and stochastic neural activity in a limb of a subject with an amputation of the limb, for example, in a method of controlling the prosthetic limb of the subject.

### CROSS REFERENCE TO RELATED APPLICATION

This application claims priority to U.S. Provisional Application No. 63/183,552, filed May 3, 2021, which is incorporated by reference in its entirety.

### FIELD

This relates to the field of peripheral nerve activity, specifically to a clinical stimulator that delivers stimulation waveforms to induce asynchronous and stochastic nerve activity, and its use.

### BACKGROUND

Traditional electrical stimulation approaches activate neural structures through the delivery of charge-balanced rectangular pulses at frequencies lower than 100 Hz. This simplistic stimulation wave elicits synchronous responses in all recruited fibers, leading to unnatural synchronicity in peripheral neural activity. This unnatural neural activity is associated with several negative effects of electrical stimulation, such as unnatural sensory percepts in amputees (e.g., tingling) and muscle fatigue during functional electrical stimulation in people with SCI or stroke. Improved stimulation approaches that elicit more natural patterns of neuronal activity are urgently needed.

### SUMMARY

Methods are disclosed for stimulating asynchronous and stochastic neural activity in a subject. In some embodiments, the method for stimulating asynchronous and stochastic neural activity in a subject comprises stimulating neurons in the subject by applying, with one or more electrodes of a neurostimulator implanted in the subject, an amplitude-modulated high-frequency (AM-HF) sinusoidal current (I) according to:

$$I = {I}_{a} sin ( 2 π {f}_{c} t ) + {I}_{b} sin ( 2 π ( {f}_{c} + {f}_{b} ) t ) \qquad ( 1 )$$

wherein Iₐ and I_{b} are current values so that the maximum current amplitude is (Iₐ+I_{b}) and the minimum current amplitude value is ∥I_{b}−Iₐ∥, f_{c} is about 800 Hz to about 20 kHz, f_{b} is about 1 Hz to about 500 Hz, and t is time. In some embodiments, the stimulation approach comprises applying an amplitude-modulated high-frequency (AM-HF) sinusoidal current (I) according to:

$$I = {I}_{p} sin ( 2 π {f}_{c} t ) + {I}_{p} sin ( 2 π ( {f}_{c} + {f}_{b} ) t ) \qquad ( 2 )$$

where 2*Iₚ is the maximum current amplitude, fc has value of about 1 Hz to about 20 kHz above, and f_{b} is about 1 Hz to about 500 Hz, and t is time.

In some embodiments, the stimulation is provided to a sensory nerve innervating an amputated limb of the subject, and wherein the stimulation is provided in an amount effective to reduce phantom limb pain in the subject.

In some embodiments, the stimulation is provided to a sensory nerve innervating an amputated limb of the subject, and is provided in response to activation of a sensor detecting movement and/or position of the amputated limb or movement and/or position of a physical or virtual prosthesis of the amputated limb.

In some embodiments, the stimulation is provided to a peripheral nerve innervating a limb of the subject with paralysis due to spinal cord injury or stroke.

In some embodiments, the stimulation is applied to a peripheral nerve innervating a limb of the subject, and the method further comprises calibrating the stimulation to induce sensations of pressure or touch in the limb.

The electrodes of the neurostimulator can be implanted at any suitable location in the subject for the intended stimulation treatment, for example, at a peripheral nerve, at the dorsal root ganglion, at the dorsal rootlets, of sensory neurons innervating a limb of the subject. In some embodiments, the neurostimulator is implanted at the lateral spinal cord adjacent to dorsal rootlets of sensory neurons innervating a limb of the subject.

Methods are also disclosed for controlling a prosthetic limb of a subject that utilize asynchronous and stochastic neural activity.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1C. Computational modelling shows that the AM-HF stimulation protocol elicits stochastic neural activity. (FIG. 1A) Schematic of the population distribution and geometry in the computational model. (FIG. 1B) Distribution of inter-spike interval values during AM-HF stimulation. The data approximate an exponential distribution, which is typical of stochastic processes. (FIG. 1C) Raster plot of the neural activity of a simulated population of fibers during AM-HF stimulation. Vertical line markers represent action potentials. Markers' location on the y axis indicates the fiber that generated the spike. Markers' location on the x-axis represents the time of occurrence of the spike.

FIGS. 2A-2D. Assessment of the AM-HF stimulation protocol using a nerve-on-a-chip in vitro system. (FIG. 2A) Firing activity following periodic square pulses stimulation (left) and periodic AM-HF stimulation (right). (FIG. 2B) Shape and features of neural responses following periodic square pulses stimulation (left) and periodic AM-HF stimulation (right). (FIG. 2C) Inter-cycle variability during AM-HF stimulation. (FIG. 2D) Histogram of inter-spike-interval of neural responses following periodic square pulses stimulation (left) and periodic AM-HF stimulation (right).

FIGS. 3A-3C. Assessment of the AM-HF stimulation protocol in an animal model. Cats were implanted with a multi-contact cuff on the sciatic nerve for stimulation as well as with microelectrode arrays in the L6 and L7 dorsal root ganglia for recording of the evoked neural response. (FIG. 3A, right) The AM-HF stimulation protocol elicited highly stochastic and asynchronous neural responses. (FIG. 3A, left) In contrast, the BioS stimulation protocol induced consistently more regular responses, which were highly synchronized with the stimulation pulses. (FIGS. 3B and 3C) Inter-spike intervals (ISI) for the BioS and AM-HF stimulation protocols.

### DETAILED DESCRIPTION

Electrical stimulation of the peripheral nerves can reproduce sensory percepts from prosthetic limbs in amputees. However, subjects often describe these sensations as unnatural. Indeed, while neurons fire with asynchronous and stochastic patterns, conventional stimulation paradigms, that use square pulses, elicit synchronous responses in all recruited axons. This generates an unnatural neural pattern. Here, a stimulation paradigm of an amplitude-modulated high-frequency sinewave is disclosed. In vitro experiments demonstrated that this approach produced asynchronous and stochastic neural activity and can be used to produce biomimetic neural patterns.

### Description of Terms

Unless otherwise noted, technical terms are used according to conventional usage. As used herein, the term “comprises” means “includes.” The singular terms “a,” “an,” and “the” include plural referents unless context clearly indicates otherwise. Although many methods and materials similar or equivalent to those described herein can be used, particular suitable methods and materials are described below. In addition, the materials, methods, and examples are illustrative only and not intended to be limiting. The scope of the claims should not be limited to those features exemplified. The word “about” indicates within five percent, unless context states otherwise. To facilitate review of the various embodiments, the following explanations of terms are provided:

Electrode: An electric conductor through which an electric current can pass. An electrode can also be a collector and/or emitter of an electric current. In some embodiments, an electrode is a solid and comprises a conducting metal as the conductive layer. Non-limiting examples of conducting metals include noble metals and alloys, such as stainless steel and tungsten. An array of electrodes refers to a device with at least two electrodes formed in any pattern. A multi-channel electrode includes multiple conductive surfaces that can independently activated to stimulate or record electrical current.

Implanting: Completely or partially placing a neurostimulator or device including a neurostimulator within a subject, for example, using surgical techniques. A device or neurostimulator is partially implanted when some of the device or neurostimulator reaches, or extends to the outside of, a subject. A neurostimulator or device can be implanted for varying durations, such as for a short-term duration (e.g., one or two days or less) or for long-term or chronic duration (e.g., one month or more).

Neural signal: An electrical signal originating in the nervous system of a subject. “Stimulating a neural signal” refers to application of an electrical current to the neural tissue of a subject in such a way as to cause neurons in the subject to produce an electrical signal (e.g., an action potential). An extracellular electrical signal can, however, originate in a cell, such as one or more neural cells. An extracellular electrical signal is contrasted with an intracellular electrical signal, which originates, and remains, in a cell. An extracellular electrical signal can comprise a collection of extracellular electrical signals generated by one or more cells.

Neurostimulator: A medical device including one or more electrodes that can be placed in electrical contact with neuronal tissue and can stimulate neural signals in the neuronal tissue. Neurostimulators typically include electrodes with conductive and non-conductive surfaces designed for contact with neuronal tissue when implanted in a subject, and can include one or more electrodes that can be independently monitored from other conductive surfaces on or off the neurostimulator for stimulating neural signals. The electrodes are linked to a stimulator suitably designed for application of various current, voltage, pulse rate, waveforms etc., for generating a neural signal in one or more neurons in proximity to the electrode or electrodes included on the device. The linkage can be by way of one or more leads, although any operable linkage capable of transmitting electrical signal from the stimulator to the electrodes may be used. The stimulator can be placed internally or externally with regard to the patient. In several embodiments, a neurostimulator for use in the disclosed methods is a pulse generator.

Phantom limb pain: An unpleasant sensory experience perceived by a subject in an amputated limb (or portion thereof) of the subject. Phantom limb pain may occur at any time after amputation, such as within the first six months following amputation, or at later times. Typical symptoms of phantom limb pain include burning sensations, sharp pains, stabbing or shooting pains, cramps, and “pins and needles” sensations. Phantom limb pain is often unresponsive to first-line treatments and can result in sleep difficulties, loss of appetite, inability to focus, impaired personal hygiene, depression, and deterioration of interpersonal relationships.

Sensory neurons: Also known as afferent neurons, sensory neurons are nerve cells within the peripheral nervous system responsible for converting stimuli from the environment of the neuron into internal electrical impulses and transmitting the impulse to the central nervous system.

Subject: Living multi-cellular vertebrate organisms, a category that includes human and non-human mammals, including non-human primates, rats, mice, guinea pigs, cats, dogs, cows, horses, and the like. Thus, the term “subject” includes both human and veterinary subjects.

Therapeutically effective amount: An amount sufficient to provide a beneficial, or therapeutic, effect to a subject or a given percentage of subjects. Therapeutically effective amounts of a therapeutic treatment can be determined in many different ways, such as assaying for a reduction in a disease or condition (such as phantom limb pain). Therapeutic agents and treatments can be administered in a single dose, or in several doses, for example daily, during a course of treatment. However, the effective amount can be dependent on the source applied, the subject being treated, the severity and type of the condition being treated, and the manner of administration.

Treating or treatment: With respect to disease or condition (e.g., phantom limb pain), either term includes (1) preventing the disease or condition, e.g., causing the clinical symptoms of the disease or condition not to develop in a subject that may be exposed to or predisposed to the disease or condition but does not yet experience or display symptoms of the disease or condition, (2) inhibiting the disease or condition, e.g., arresting the development of the disease or condition or its clinical symptoms, or (3) relieving the disease or condition, e.g., causing regression of the disease or condition or its clinical symptoms.

Additional terms commonly used in molecular genetics can be found in Krebs et al. (eds.), Lewin's genes XII, published by Jones & Bartlett Learning, 2017, The Encyclopedia of Cell Biology and Molecular Medicine, published by Wiley-VCH in 16 volumes, 2008; and other similar references.

### Methods for Inducing Asynchronous and Stochastic Neural Activity

Traditional electrical stimulation paradigms activate neural structures through the use of charge-balanced rectangular pulses at frequencies lower than 100 Hz. This simplistic approach elicits synchronous responses in all recruited fibers. This unnatural synchronicity in peripheral neural activity is believed to be responsible for several negative effects of electrical stimulation, such as the production of unnatural sensory percepts in amputees (e.g., tingling) (Tan et al., 2014, Sci. Transl. Med. 6 257ra138, and or muscle fatigue during functional electrical stimulation in people with SCI or stroke (Gorgey, et al. Journal of Orthopaedic & Sports Physical Therapy 39.9 (2009): 684-692). A stimulation paradigm is disclosed herein that can induce more natural, asynchronous and stochastic neural activity.

In some embodiments, the stimulation approach comprises applying an amplitude-modulated high-frequency (AM-HF) sinusoidal current (I) according to:

$$I = {I}_{a} sin ( 2 π {f}_{c} t ) + {I}_{b} sin ( 2 π ( {f}_{c} + {f}_{b} ) t ) \qquad ( 1 )$$

wherein Iₐ and I_{b} are current values so that the maximum current amplitude is (Iₐ+I_{b}) and the minimum current amplitude value is ∥I_{b}−Iₐ∥, f_{c} is about 800 kHz to about 20 kHz, f_{b} is about 1 Hz to about 500 Hz, and t is time, resulting in a sinusoid at the selected fc frequency, with an envelope that beats at frequency fb.

In some embodiments, the stimulation approach comprises applying an amplitude-modulated high-frequency (AM-HF) sinusoidal current (I) according to:

$$I = {I}_{p} sin ( 2 π {f}_{c} t ) + {I}_{p} sin ( 2 π ( {f}_{c} + {f}_{b} ) t ) \qquad ( 2 )$$

where 2*Iₚ is the maximum current amplitude, fc has value of about 1 kHz to about 20 kHz above, and f_{b} is about 1 Hz to about 500 Hz, and t is time, resulting in a sinusoid at the selected f_{c} frequency, with an envelope that beats at frequency fb. In some embodiments, the maximum current amplitude is Iₚ.

In a specific non-limiting example, fc has value of about 1000 Hz and f_{b} is about 10 Hz, resulting in an about 1000 Hz sinusoid with an envelope that beats at about 10 Hz.

In some embodiments, fc can have a value of about 800 Hz to about 8 kHz, or 900 Hz, about 1 kHz to about 20 kHz. In further embodiments, fc has a value of about 1 kHz to about 5 kHz, or about 1 kHz to about 10 kHz, or about 1 kHz to about 15 kHz. In some embodiments, fc has a value of about 2 kHz to about 20 kHz, about 5 kHz to about 20 kHz, about 10 kHz to about 20 kHz, or about 15 kHz to about 20 kHz. In other embodiments, fc has a valuate of about 1, 2, 3, 4, 5, 6, 7, 8, 9, 10, 11, 12, 13, 14, 15, 6, 17, 18, 19 or 20 KHz.

In other embodiments, fb can have a value of about 1 Hz to about 500 Hz. In yet other embodiments, fb is about 5 Hz to about 500 Hz, about 50 Hz to about 500 Hz, about 100 Hz to about 500 Hz, about 150 Hz to about 500 Hz, about 200 Hz to about 500 Hz, about 250 Hz to about 500 Hz, about 300 Hz to about 500 Hz, about 350 Hz to about 500 Hz, about 400 Hz to about 500 Hz, or about 450 Hz to about 500 Hz. In more embodiments, fb is about 1 Hz to about 500 Hz, about 1 Hz to about 400 Hz, about 1 Hz to about 300 Hz, about 1 Hz to about 200 Hz, about 1 Hz to about 100 Hz, about 1 Hz to about 50 Hz, about 1 Hz to about 10 Hz or about 1 Hz to about 5 Hz.

The outcome of this stimulation protocol differs substantially from the neural activity elicited with square pulses or prior methods of eliciting asynchronous neural activity. When a square pulse is delivered, all neural structures recruited respond simultaneously. Conversely, when the AM-HF pulse is delivered, single fibers' responses occur asynchronously with different latencies during each amplitude modulation cycle.

The disclosed AM-HF stimulation protocol can be used to induce asynchronous and stochastic neural activity in a subject for any suitable neural stimulation application, including CNS applications, and peripheral applications, such as sensory applications. Inducing asynchronous neural activity facilitates the production of more naturalistic neural response and can be used, for example, in upper and lower limb amputees to restore sensory feedback that feels natural to the subject, or during functional electrical stimulation in patients with spinal cord injury or stroke. In some embodiments, the disclosed AM-HF stimulation protocol is provided to a peripheral nerve innervating a limb of the subject with complete or partial paralysis due to spinal cord injury or stroke, for example in an amount effective to cause reflexive muscle contraction in the paralyzed limb.

Any type of neurostimulator that would benefit from the AM-HF stimulation protocol described herein can be utilized with the disclosed methods. The electrodes of the neurostimulator can take any suitable form for the intended use, such as a nerve cuff, an electrode array, a microelectrode array (e.g., Utah and Michigan microelectrode arrays), a deep brain stimulator, a peripheral nerve stimulator, or a neural electrode. In several embodiments, the neurostimulator includes more than one electrode, such as an array of electrodes. In additional embodiments, a device is provided that can include one or more neural probes, each of which can include one or more electrodes.

For example, any neurostimulator suitable for stimulating the peripheral neurons, such as sensory neurons innervating the affected limb, such as peripheral nerves in the limb with the phantom limb pain as well as the DRG, dorsal rootlets of one or more sensory neurons innervating a limb of a subject, and/or lateral spinal cord adjacent to the dorsal rootlets, can be used in the method provided herein. The neurostimulator includes a device for generating electrical current (the stimulator) connected to one or more electrodes suitable for conducting the current from the stimulator to the appropriate anatomical location in the subject. Typically, the stimulator is suitably designed for application of various current, voltage, pulse rate, waveforms etc., via the electrodes for generating neuronal activity in one or more neurons in proximity to the individual electrodes. In some embodiments, the neurostimulator is a pulse generator. In several embodiments, the neurostimulator is a commercially available FDA approved spinal cord stimulator placed in the epidural space. A more complex device with a higher density of electrode contacts and shapes and sizes that better conform to the anatomical target may also be implanted to function as a stimulator.

In several embodiments, the neurostimulator includes integrated circuitry to control the functions of the neurostimulator, including generation and application of electrical signals (via one or more channels of the electrodes implanted in the subject) to stimulate sensory neurons at the target location in the subject in response to activation of one or more sensors as described herein. The integrated circuitry can comprise and/or be included within a controller (e.g., processor) for controlling the operations of the neurostimulator, including stimulating, signal transmission, charging and/or using energy from a battery for powering the various components of the device, and the like. Typically, the neurostimulator includes a pulse generator that provides stimulation energy in programmable patterns adapted for direct stimulation of neurons.

In some embodiments, the method further includes implanting the electrodes of the neurostimulator in the subject. The electrodes can be implanted at any location in the subject that is suitable for stimulation of neural signals. The electrodes of the neurostimulator are implanted in the subject within a suitable distance of target neurons, such as peripheral neurons that innervate a limb of the subject and/or dorsal rootlets and/or lateral spinal cord of one or more sensory neurons innervating a limb of the subject. Any appropriate method may be used to implant the electrodes of the neurostimulator at an appropriate anatomical location in the subject. In several embodiments, the electrodes of the neurostimulator are tunneled percutaneously and secured in place with tape or suture in the subject. The electrodes may be steered laterally under fluoroscopic guidance to target the dorsal rootlets and the lateral spinal cord, for example, using a stylet.

The implanted neurostimulator may remain in place for any suitable time period (such as about one month, about two months, about three months, about six months, about one year, or longer). In some embodiments, the electrodes remain implanted in the subject for the duration of time that the method provides a therapeutic benefit to the subject.

In several embodiments, the method further comprises calibrating the applied stimulation parameters to induce sensations of pressure, touch, or joint movement in the subject. During calibration, subjects may be asked to report sensations experienced in response to the stimulation, and the stimulation parameters and/or electrode channels varied until the subject experiences sensations (such as sensations of pressure, touch, or joint movement).

### Treating Phantom Limb Pain and Restoring Sensory Feedback

In some embodiments, the disclosed AM-HF stimulation protocol is used to treating phantom limb pain in a subject with or at risk of such pain. The method comprises providing a therapeutically effective amount of stimulation to peripheral neurons that innervate the limb of the subject and/or DRG, dorsal rootlets, or lateral spinal cord adjacent to the dorsal rootlets, of sensory neurons innervating a limb of the subject with the phantom limb pain. The stimulation is provided with a clinical stimulator that can deliver stimulation waveforms able to induce asynchronous and stochastic neural activity.

Any neurostimulator suitable for stimulating the peripheral neurons, such as sensory neurons innervating the affected limb, such as peripheral nerves in the limb with the phantom limb pain as well as the DRG, dorsal rootlets of one or more sensory neurons innervating a limb of a subject, and/or lateral spinal cord adjacent to the dorsal rootlets, can be used in the method provided herein.

The electrodes of the neurostimulator can have any form appropriate for stimulating neural signals in the peripheral neurons that innervate the limb of the subject and/or dorsal rootlets of one or more sensory neurons innervating a limb of a subject, and/or lateral spinal cord adjacent to the dorsal rootlets. In some embodiments, multi-channel electrodes are used. For example, the individual channels of the electrode can be calibrated to generate neural signals at a desired location in the subject (such as neural signals that induce sensations of pressure, touch, or joint movement at the location of the phantom limb pain or the diabetic neuropathy pain in the subject).

The electrodes of the neurostimulator are implanted in the subject within a suitable distance of the peripheral neurons that innervate the limb of the subject and/or dorsal rootlets and/or lateral spinal cord of one or more sensory neurons innervating a limb of the subject with the phantom limb pain or the diabetic neuropathy pain. In some embodiments, the electrodes are located in the epidural space above the dorsal rootlets of one or more sensory neurons innervating a limb of the subject with the phantom limb pain or the diabetic neuropathy pain. In some embodiments, the electrodes are located in the epidural space above the lateral spinal cord adjacent to the dorsal rootlets of one or more sensory neurons innervating a limb of the subject.

The electrodes can be implanted adjacent to the dorsal rootlets and/or lateral spinal cord at any appropriate position along the spinal cord, depending on the limb of the subject affected. Lumbar L2, L3, L4, L5, and/or sacral S1 are known to contain sensory neurons receiving signals from the lower extremities and can be targeted for stimulation using the method provided herein. Cervical DRG C3, C4, C5, C6, C7, thoracic DRG T1 are known to contain sensory neurons receiving signals from the upper extremities and can be targeted for stimulation using the method provided herein.

In several embodiments, the one or more electrodes are activated to stimulate neural signals in the peripheral neurons that innervate the limb of the subject and/or dorsal rootlets of one or more sensory neurons innervating a limb of a subject, and/or lateral spinal cord adjacent to the dorsal rootlets, in response to activation of a sensor detecting movement of the limb or movement of a physical or virtual prosthesis of the limb.

In several embodiments, when the subject moves the limb (or a prosthetic of the limb or portion thereof), this movement may be detected by a sensor capable of capturing such limb movement. The sensors may be placed at a suitable location to detect movements of the limb or prosthesis, for example, in a shoe or in a glove.

In embodiments involving a prosthetic limb (or portion thereof), any sensor suitable for detecting movement of the prosthetic may be used. The sensor may be located in or on the prosthetic, or in or on a stump of the limb to which the prosthetic is attached. In embodiments where the sensor is located in or on a limb of the subject (for example, in the case of a subject with diabetic neuropathy pain), any sensor suitable for detecting movement of the prosthetic may be used. Non-limiting examples of sensors for detecting movement of a limb or prosthetic limb include a gyroscope, an electrogoniometer, a textile piezoresistive sensor, or a pressure sensor located in or on the prosthesis, or in or on a limb, or stump of the limb to which the prosthetic is attached.

In some embodiments, the stimulation is applied in response to movement of a virtual prosthesis. The subject observes the virtual limb or prosthesis, the movement of which is used to trigger activation of the neurostimulator.

The applied stimulus parameters can vary depending the particular subject and desired outcome. In several embodiments, the stimulus parameters are calibrated for the particular subject to be treated with the disclosed method. For example,

In some embodiments, the intensity of the stimulation (and resulting sensation) can be calibrated to correlate with the level of activity detected by the sensor detecting movement of the limb or movement of a physical or virtual prosthesis of the limb in the subject. For example, the stimulation parameters applied when the subject puts all their body weight on a prosthetic limb would elicit a more intense sensation of pressure or touch than when the subject places only part of their bodyweight on the prosthetic limb.

In several embodiments, the neurostimulator provides patterns of electrical stimulation to the peripheral neurons that innervate the limb of the subject and/or dorsal rootles and lateral spinal cord that elicit pressure, touch, or joint movement sensations at the location of the phantom limb pain or diabetic neuropathy pain in the subject. For example, a microprocessor may be provided in conjunction with the neurostimulator that is programmed to accept signals produced by sensors in the prosthetic or virtual limb and transduce the signals to electrical signals sent via the implanted electrodes to the peripheral neurons that innervate the limb of the subject and/or dorsal rootlets or lateral spinal cord adjacent to the dorsal rootlets. In some embodiments, signals from the sensor(s) in the prosthetic limb may be sent directly from a transmitter in the prosthetic limb to a receiver implanted in the subject and linked to the neurostimulator.

In several embodiments, the method further comprises calibrating the stimulation parameters applied to the peripheral neurons that innervate the limb of the subject and/or dorsal rootlets of one or more sensory neurons innervating a limb of a subject, and/or lateral spinal cord adjacent to the dorsal rootlets, to induce sensations of pressure, touch, or joint movement in the subject. For example, in some embodiments, an amputee receives the sensation of a fingertip touching something if a touch sensor on a fingertip of the prosthetic limb is activated. In some embodiments, a microprocessor is included in or with the neurostimulator that can be programmed to facilitate this type of sensor-activated stimulation.

During calibration, subjects may be asked to report sensations experienced in response to the stimulation, and the stimulation parameters and/or electrode channels varied until the subject experiences sensations (such as sensations of pressure, touch, or joint movement). Additionally, the intensity of the stimulation (and resulting sensation) can be calibrated to correlate with the level of activity detected by the sensor detecting movement of the limb or movement of a physical or virtual prosthesis of the limb in the subject. For example, the stimulation parameters applied when the subject puts all their body weight on a prosthetic limb would provide a more intense sensation of pressure or touch than when the subject places only part of their bodyweight on the prosthetic limb. Additionally, the subject can be evaluated for an overall level pain after each stimulus presentation or after a series of presentations. In a non-limiting embodiment, pain level is evaluated based on the McGill pain score.

### Control of a Prosthetic Limb of a Subject

In some embodiments, the disclosed AM-HF stimulation protocol is used in a method of controlling a prosthetic limb of a subject. The method comprises providing a therapeutically effective amount of stimulation to peripheral neurons that innervate the limb of the subject and/or dorsal rootlets, or lateral spinal cord adjacent to the dorsal rootlets, of sensory neurons innervating an amputated limb of the subject with a prosthesis. The stimulation is applied using the disclosed AM-HF stimulation protocol disclosed herein. The stimulation is provided with one or more electrodes of a neurostimulator that are implanted within electrical contact with the peripheral neurons (such as at the dorsal rootlets or the lateral spinal cord adjacent to the dorsal rootlets) in the subject. The one or more electrodes are activated to provide the stimulation in response to activation of a sensor detecting movement and/or position of the limb or movement the prosthesis or movement of a virtual prosthesis of the limb. Coordination of the stimulation with the activation of the sensor increases control of the prosthesis by the subject.

Any appropriate subject with a prosthetic limb can be treated with the method provided herein. The prosthetic can be an upper or lower body prosthetic, such as an above or below the elbow arm prosthetic, or an above or below the knee leg prosthetic.

The method can be in initiated at any time post-amputation to improve control of the prosthetic by the subject. In some embodiments, the method provided herein is implemented as soon as possible following limb amputation (or even before), so as to maximally arrest cortical changes subsequent to amputation.

In some embodiments, the method provided herein increases control of the prosthetic limb by the subject by at least 20% (such as at least 30%, at least 40%, at least 50%, at least 60%, at least 70%, at least 80%, at least 90%) relative to before the treatment as measured by any appropriate evaluation metric, such as a balance or strength metric (e.g., a sensory organization test).

In some embodiments, the method provided herein increases postural balance and stability by the subject by at least 20% (such as at least 30%, at least 40%, at least 50%, at least 60%, at least 70%, at least 80%, at least 90%) relative to before the treatment as measured by any appropriate evaluation metric, such as a balance or strength metric (e.g., a sensory organization test).

The subject has one or more electrodes of a neurostimulator as implanted within electrical contact of the peripheral neurons that innervate the limb of the subject and/or at the dorsal rootlets, and/or the lateral spinal cord adjacent to the dorsal rootlets, of one or more sensory neurons innervating the limb of the subject with the prosthetic. The neurostimulator, electrodes, and target location of the electrodes used in the method of treating phantom limb pain as described herein can also be used in the disclosed method of increasing control of a prosthetic limb.

The one or more electrodes are activated to stimulate neural signals in the peripheral neurons that innervate the limb of the subject and/or dorsal rootlets of one or more sensory neurons innervating a limb of a subject, and/or lateral spinal cord adjacent to the dorsal rootlets, in response to activation of a sensor detecting movement and/or position of a physical or virtual prosthesis of the amputated limb. In several embodiments, when the subject moves the limb (or a prosthetic of the limb or portion thereof), this movement may be detected by a sensor capable of capturing such limb movement. The sensors may be placed at a suitable location to detect movements of the limb or prosthesis, for example, in a shoe or in a glove.

Any sensor suitable for detecting movement and/or position of the prosthetic may be used. The sensor may be located in or on the prosthetic, or in or on a stump of the limb to which the prosthetic is attached. Non-limiting examples of sensors for detecting movement of a prosthetic limb include a gyroscope, an electrogoniometer, a textile piezoresistive sensor, or a pressure sensor located in or on the prosthesis, or in or on a stump of the limb to which the prosthetic is attached. In some embodiments, the stimulation is applied in response to movement of a virtual prosthesis or limb. The subject observes the virtual limb or prosthesis, the movement of which is used to trigger activation of the neurostimulator.

The applied stimulus parameters can vary depending the particular subject and desired outcome. In several embodiments, the stimulus parameters are calibrated for the particular subject to be treated with the disclosed method. In some embodiments, varying the electrical current pattern applied to the electrodes creates specific stimulation pattern to be delivered to the stimulation target. Stimulation parameters may be modulated including stimulus amplitude, pulse width, and frequency in order to produce natural sensations in subjects. This may be achieved by providing recurring stimulation that mimic the natural firing patterns of sensory afferent neurons. These natural sensations may be effective in increasing the control of the prosthetic limb by the subject. In some embodiments, recurring trains of stimulus pulses may be delivered to the anatomical targets. Further, the duration and frequency of stimulation can be varied as needed to optimize therapeutic outcome.

In several embodiments, stimulation parameters are selected that elicit appropriate focal sensations of touch, pressure, joint movement, proprioception, and/or kinesthesia in the missing limb in the subject.

In some embodiments, the intensity of the stimulation (and resulting sensation) can be calibrated to correlate with the level of activity detected by the sensor detecting movement of the limb or movement of a physical or virtual prosthesis of the limb in the subject. For example, the stimulation parameters applied when the subject puts all their body weight on a prosthetic limb would elicit a more intense sensation of pressure or touch than when the subject places only part of their bodyweight on the prosthetic limb.

In several embodiments, stimulating the peripheral neurons that innervate the limb of the subject and/or dorsal rootlets or the lateral spinal cord does not induce paresthesia in the amputated limb.

In some embodiments, a particular pattern of stimulation, which may be person-specific, will be more effective than others at increasing control of the prosthetic by the subject. In some embodiments, a pattern of signals approximating the train of signals received from a normal, innervated limb for communicating sensations of pressure, touch, joint movement, proprioception, and/or kinesthesia to the cortex is used. In some embodiments, the neurostimulator may be programmed to optimize such stimulation patterns, or the choice of stimulation patterns may be controlled by the subject or a health care provider. For example, subject or health care provider may adjust the amplitude and frequency of signals, for example, and also may select which channel (i.e., electrode) transmits which signal, to optimize signal pattern.

In several embodiments, the neurostimulator provides patterns of electrical stimulation to the peripheral neurons that innervate the limb of the subject and/or dorsal rootles and lateral spinal cord that elicit pressure, touch, joint movement, proprioceptive, and/or kinesthetic sensations in the amputated limb. For example, a microprocessor may be provided in conjunction with the neurostimulator that is programmed to accept signals produced by sensors in the prosthetic or virtual limb and transduce the signals to electrical signals sent via the implanted electrodes to the peripheral neurons that innervate the limb of the subject and/or dorsal rootlets or lateral spinal cord adjacent to the dorsal rootlets. In some embodiments, signals from the sensor(s) in the prosthetic limb may be sent directly from a transmitter in the prosthetic limb to a receiver implanted in the subject and linked to the neurostimulator.

In several embodiments, the method further comprises calibrating the stimulation parameters applied to the peripheral neurons that innervate the limb of the subject and/or dorsal rootlets of one or more sensory neurons innervating a limb of a subject, and/or lateral spinal cord adjacent to the dorsal rootlets, to induce sensations of pressure, touch, joint movement, proprioception, and/or kinesthesia in the amputated limb. For example, in some embodiments, an amputee receives the sensation of a fingertip touching something if a touch sensor on a fingertip of the prosthetic limb is activated. In some embodiments, a microprocessor is included in or with the neurostimulator that can be programmed to facilitate this type of sensor-activated stimulation. It is believed that inducing such sensations in the amputated limb of the subject leads to a superior increase in control of a corresponding prosthetic.

During calibration, subjects may be asked to report sensations experienced in response to the stimulation, and the stimulation parameters and/or electrode channels varied until the subject experiences sensations (such as sensations of pressure, touch, joint movement, proprioception, and/or kinesthesia) in the amputated limb. Additionally, the intensity of the stimulation (and resulting sensation) can be calibrated to correlate with the level of activity detected by the sensor detecting movement of the limb or movement of a physical or virtual prosthesis of the limb in the subject. For example, the stimulation parameters applied when the subject puts all their body weight on a prosthetic limb would provide a more intense sensation of pressure or touch than when the subject places only part of their bodyweight on the prosthetic limb. Additionally, the subject can be evaluated for physical control of the prosthetic after each stimulus presentation or after a series of presentations using any suitable evaluation criteria, such as balance, dexterity, and/or strength.

### Additional Description

Clause 1. A method for stimulating asynchronous and stochastic neural activity in a subject, comprising:
stimulating neurons in the subject by applying, with one or more electrodes of a neurostimulator implanted in the subject, an amplitude-modulated high-frequency (AM-HF) sinusoidal current (I) according to:

$$I = {I}_{a} sin ( 2 π {f}_{c} t ) + {I}_{b} sin ( 2 π ( {f}_{c} + {f}_{b} ) t ) \qquad ( 1 )$$

wherein Iₐ and I_{b} are current values so that the maximum current amplitude is (Iₐ+I_{b}) and the minimum current amplitude value is ∥I_{b}−Iₐ∥, f_{c} is about 800 Hz to about 20 kHz, f_{b} is about 1 Hz to about 500 Hz, and t is time.

Clause 2. The method of Clause 1, wherein the AM-HF sinusoidal current (I) is according to:

$$I = {I}_{p} sin ( 2 π {f}_{c} t ) + {I}_{p} sin ( 2 π ( {f}_{c} + {f}_{b} ) t ) \qquad ( 2 )$$

wherein 2*Iₚ is the maximum current amplitude, f_{c} is about 1 kHz to about 20 kHz, f_{b} is about 1 Hz to about 500 Hz, and t is time.

Clause 3. The method of Clause 1 or Clause 2, wherein the stimulation is applied to a peripheral nerve innervating a limb of the subject.

Clause 4. The method of any one of Clauses 1-3, wherein the stimulation is provided to a sensory nerve innervating an amputated limb of the subject, and wherein the stimulation is provided in an amount effective to reduce phantom limb pain in the subject.

Clause 5. The method of any one of Clauses 1-3, wherein the stimulation is provided to a sensory nerve innervating an amputated limb of the subject, and wherein the stimulation is provided in response to activation of a sensor detecting movement and/or position of the amputated limb or movement and/or position of a physical or virtual prosthesis of the amputated limb.

Clause 6. The method of Clause 5, wherein the sensor is any one of a gyroscope, an electrogoniometer, a textile piezoresistive sensor, or a pressure sensor located in or on the limb or the prosthesis of the subject

Clause 7. The method of any one of Clauses 5-6, wherein the sensor detects pressure at the fingertips of a prosthetic hand or in the sole of a prosthetic foot.

Clause 8. The method of any one of Clauses 5-7, wherein the sensor detects a position of the limb, the stump of the limb, the prosthesis, or the virtual prosthesis.

Clause 9. The method of Clause 8, wherein the virtual prosthesis of the limb is a computer-generated image of the limb and the movement of the computer-generated image of the limb is observed or controlled by the subject.

Clause 10. The method of any one of Clauses 1-3, wherein the stimulation is provided to a peripheral nerve innervating a limb of the subject with complete or partial paralysis due to spinal cord injury or stroke.

Clause 11. The method of Clause 10, wherein the stimulation is provided in an amount effective to cause reflexive muscle contraction in the limb of the subject with complete or partial paralysis due to spinal cord injury or stroke.

Clause 12. The method of any one of the prior Clauses, wherein the stimulation is applied to a peripheral nerve innervating a limb of the subject, and the method further comprises calibrating the stimulation to induce sensations of pressure or touch in the limb.

Clause 13. The method of any one of Clauses 3-12, wherein the neurostimulator is implanted at the dorsal rootlets of sensory neurons innervating the limb of the subject.

Clause 14. The method of any one of Clauses 3-12, wherein the neurostimulator is implanted at the lateral spinal cord adjacent to the dorsal rootlets of the sensory neurons innervating the limb of the subject.

Clause 15. The method of any one of Clauses 3-12, wherein the neurostimulator is implanted on peripheral neurons innervating the limb of the subject.

Clause 16. The method of any one of the prior Clauses, wherein the electrodes of the neurostimulator are in the form of a nerve cuff, a microelectrode array, a deep brain stimulator, or a spinal probe.

Clause 17. The method of any one of the prior Clauses, wherein the neurostimulator is an external or implanted pulse generator.

Clause 18. The method of any one of the prior Clauses, further comprising implanting the neurostimulator in the subject.

Clause 19. A method for providing asynchronous and stochastic neural activity in a limb of a subject with an amputation of the limb, comprising:
providing a therapeutically effective amount of stimulation to peripheral neurons that innervate the limb of the subject and/or dorsal rootlets, or lateral spinal cord adjacent to the dorsal rootlets, of one or more sensory neurons that innervate the limb of the subject, wherein:
the stimulation is provided with one or more electrodes of a neurostimulator that are implanted at the peripheral neurons that innervate the limb of the subject and/or dorsal rootlets or the lateral spinal cord adjacent to the dorsal rootlets of the one or more sensory neurons innervating the limb of the subject;
the one or more electrodes are activated to provide the stimulation in response to activation of a sensor detecting movement and/or position of the limb or movement and/or position of a physical or virtual prosthesis of the limb; and
wherein the stimulation is amplitude-modulated high-frequency (AM-HF) sinusoidal current (I) according to:

$$I = {I}_{a} sin ( 2 π {f}_{c} t ) + {I}_{b} sin ( 2 π ( {f}_{c} + {f}_{b} ) t ) \qquad ( 1 )$$

wherein Iₐ and I_{b} are current values so that the maximum current amplitude is (Iₐ+I_{b}) and the minimum current amplitude value is ∥I_{b}−Iₐ∥, f_{c} is about 800 Hz to about 20 kHz, f_{b} is about 1 Hz to about 500 Hz, and t is time.

Clause 20. A method for control of a prosthetic limb of a subject, comprising:
providing a therapeutically effective amount of stimulation to peripheral neurons that innervate the limb of the subject and/or dorsal rootlets, or lateral spinal cord adjacent to the dorsal rootlets, of sensory neurons innervating an amputated limb of the subject, wherein the subject uses a prosthesis of the amputated limb; and wherein:
the stimulation is provided with one or more electrodes of a neurostimulator that are implanted at peripheral neurons that innervate the limb of the subject and/or the dorsal rootlets or the lateral spinal cord adjacent to the dorsal rootlets of the sensory neurons innervating the amputated limb;
the one or more electrodes are activated to provide the stimulation in response to activation of a sensor detecting movement and/or position of a stump of the limb or the prosthesis or in response to movement and/or position of a virtual prosthesis of the limb;
wherein the stimulation is amplitude-modulated high-frequency (AM-HF) sinusoidal current (I) according to:

$$I = {I}_{a} sin ( 2 π {f}_{c} t ) + {I}_{b} sin ( 2 π ( {f}_{c} + {f}_{b} ) t ) \qquad ( 1 )$$

wherein Iₐ and I_{b} are current values so that the maximum current amplitude is (Iₐ+I_{b}) and the minimum current amplitude value is ∥I_{b}−Iₐ∥, f_{c} is about 800 Hz to about 20 kHz, f_{b} is about 1 Hz to about 500 Hz, and t is time; and
and wherein the stimulation controls the prosthesis of the limb by the subject.

### EXAMPLES

The following examples are provided to illustrate particular features of certain embodiments, but the scope of the claims should not be limited to those features exemplified.

### Example 1

Electrical stimulation of the peripheral nerves can reproduce sensory percepts from prosthetic limbs in amputees. However, subjects often describe these sensations as unnatural. Indeed, while neurons fire with asynchronous and stochastic patterns, conventional stimulation paradigms, that use square pulses, elicit synchronous responses in all recruited axons. This generates an unnatural neural pattern. This example provides results of computational, nerve-on-a-chip, and in vivo assays demonstrating an amplitude-modulated high-frequency stimulation (AM-HF) protocol that elicits asynchronous and stochastic activation of neural signals and provides a protocol to stimulate biomimetic neural patterns.

### Computational Modeling

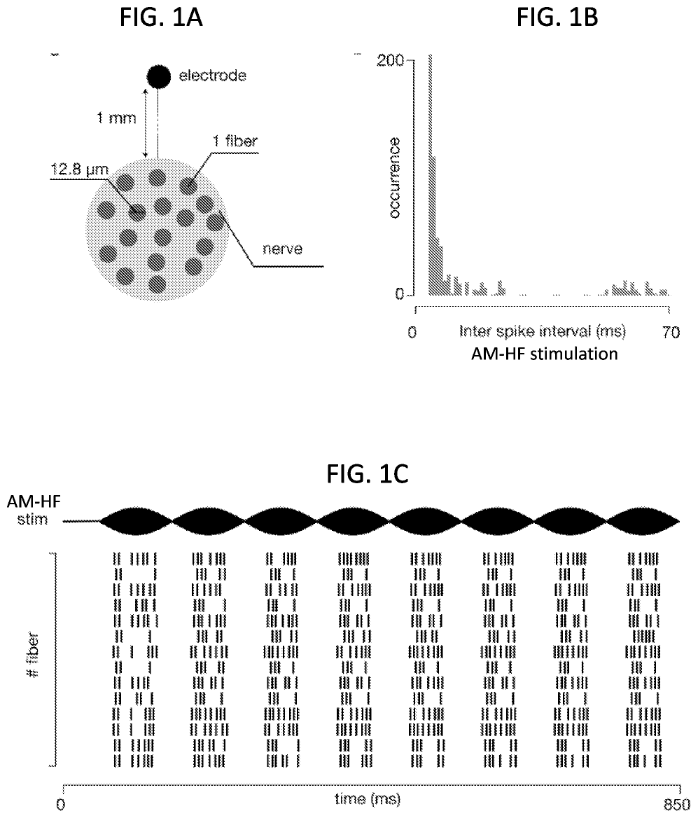

First, a computation model study of the AM-HF stimulation protocol was conducted (FIG. 1) using a computation system derived from Mirzakhalili et al. (“Biophysics of Temporal Interference Stimulation,” Cell Systems, 11, 557-572, 2020). FIG. 1A shows a schematic of the population distribution and geometry in the computational model. For the stimulation model, a charge-balanced, AM-HF sinusoidal current was delivered at 1000 Hz, as defined below,

$$I = {I}_{p} sin ( 2 π {f}_{c} t ) + {I}_{p} sin ( 2 π ( {f}_{c} + {f}_{b} ) t ) \qquad ( 1 )$$

where 2*Iₚ is the maximum current amplitude, fc has value 1000 Hz and f_{b} is 10 Hz, resulting in a 1000 Hz sinusoid with an envelope that beats at 10 Hz. The results show an exponential distribution of inter-spike interval during stimulation (FIG. 1B), which is typical of stochastic processes. FIG. 1C shows a Raster plot of the neural activity of a simulated population of fibers during AM-HF stimulation, showing asynchronous activity typical of stochastic neural patterns.

### Nerve-On-a-Chip

Second, a nerve-on-a-chip platform was used to record and stimulate explanted rat dorsal rootlets. The nerve-on-a-chip platform is described in Gribi et al. (“A microfabricated nerve-on-a-chip platform for rapid assessment of neural conduction in explanted peripheral nerve fibers,” Nat. Commun., 9, 1-10, 2018). Similar to the computational modeling, a charge-balanced, AM-HF sinusoidal current was delivered to the explanted neural tissue at 1000 Hz, as defined below,

$$I = {I}_{p} sin ( 2 π {f}_{c} t ) + {I}_{p} sin ( 2 π ( {f}_{c} + {f}_{b} ) t ) \qquad ( 1 )$$

where 2*Iₚ is the maximum current amplitude, fc has value 1000 Hz and f_{b} is 10 Hz, resulting in a 1000 Hz sinusoid with an envelope that beats at 10 Hz. For comparison, squared charge-balanced pulses were delivered at frequency f_{b}=10 Hz.

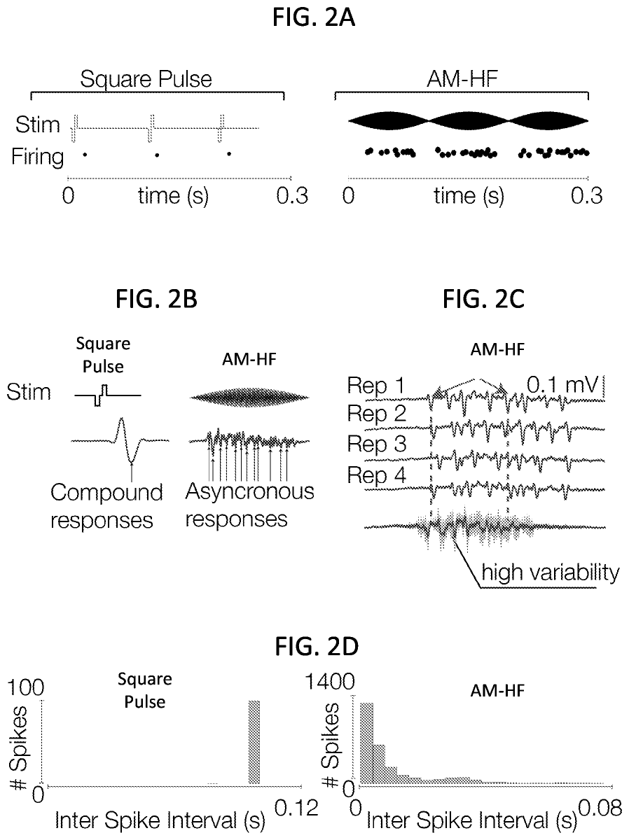

FIG. 2A shows an example of the firing pattern during each stimulation type. When a square pulse was delivered, all fibers responded simultaneously: the recorded potential (FIG. 2B) shows a large compound action potential, which is the summation of each fiber's action potential. Conversely, when the AM-HF pulse was delivered, single fibers' responses occurred asynchronously with different latencies during each amplitude modulation cycle (FIGS. 2B, 2C). The voltage profile in FIG. 2B shows that multiple neural responses follow each other closely in time during the AM-HF pulse, spread throughout the duration of the whole stimulus cycle. To verify that the neural activity induced by AM-HF stimulation was stochastic, inter-spike intervals (ISI) within each stimulation cycle were computed. FIG. 2D shows that ISIs during AM-HF stimulation follow an exponential distribution, which is a distinctive feature for stochastic processes.

These nerve-on-a chip assays show that the AM-HF stimulation protocol described herein can be used for inducing asynchronous and stochastic neural activity, facilitating activation of more naturalistic stimulation patterns than prior stimulation protocols.

### Assessment in an Animal Model

Pre-clinical assays were performed using a cat model to interrogate the AM-HF stimulation protocol in an in vivo environment. All experiments were performed under anesthesia. Animals were implanted with a multi-contact cuff on the sciatic nerve for stimulation as well as with microelectrode arrays (Blackrock Microsystems) in the L6 and L7 dorsal root ganglia for recording of the evoked neural response. Similar to the computational and nerve-on-a-chip studies, a charge-balanced, AM-HF sinusoidal current was delivered to the sciatic nerve via the nerve cuff at 1000 Hz, as defined below,

$$I = {I}_{p} sin ( 2 π {f}_{c} t ) + {I}_{p} sin ( 2 π ( {f}_{c} + {f}_{b} ) t ) \qquad ( 1 )$$

where 2*Iₚ is the maximum current amplitude, fc has value 1000 Hz and f_{b} is 10 Hz, resulting in a 1000 Hz sinusoid with an envelope that beats at 10 Hz. Resulting neural signals were recorded with the implanted microelectrode arrays at the L6 and L7 DRGs. For comparison, the biomimetic stimulation (BioS) strategy of Formento et al. (“A biomimetic electrical stimulation strategy to induce asynchronous stochastic neural activity,” J Neural Engineer, 17(4):046019, 2020) was also assessed.

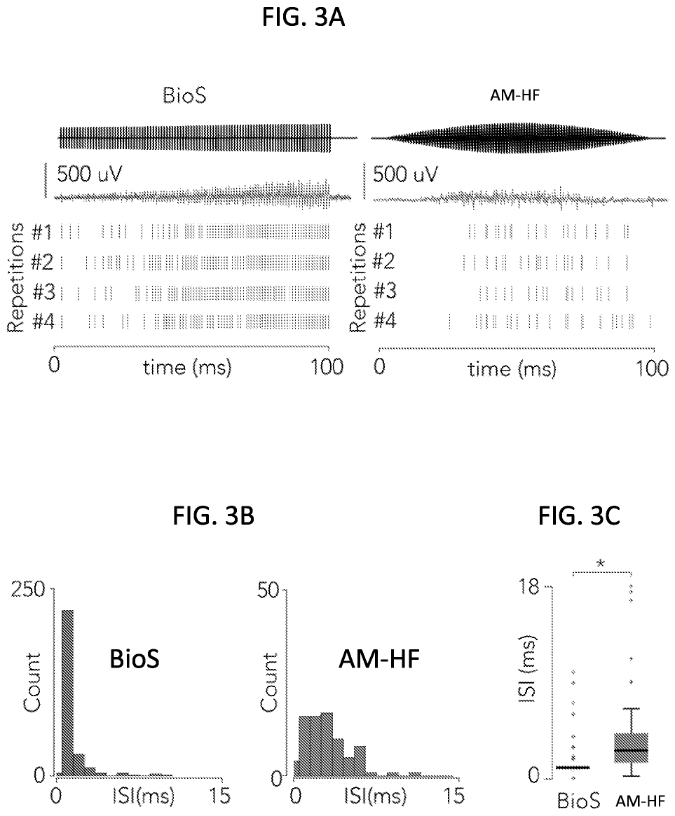

Consistent with the results of the computational and nerve-on-a-chip models, the AM-HF protocol elicited highly stochastic and asynchronous neural responses in the cat model (FIG. 3A, right). In contrast, the BioS stimulation protocol induced consistently more regular responses, which were highly synchronized with the stimulation pulses (FIG. 3A, left). In order to quantify these effects, the inter-spike intervals (ISI) were computed during the two types of stimulation. While BioS induced very fast and regular ISI spiking (1.4±1.3 ms, mean and standard deviation), the AM-HF protocol induced a more sparse and variable ISI spiking (3.6±3.4 ms, mean and standard deviation) (FIGS. 3B,3C, Wilcoxon test, p<0.05).

These results show that the AM-HF stimulation protocol described herein can achieve asynchronous and stochastic activation of neurons, and that the patterns of activity are substantially less synchronized than those evoked by either square stimulus pulses or prior techniques designed to desynchronize the response to stimulation. The AM-HF stimulation protocol provides for more natural recruiting and activation of neural fibers than previously available stimulation protocols, and can be used in stimulation.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for stimulating asynchronous and stochastic neural activity in a subject, comprising:
stimulating neurons in the subject by applying, with one or more electrodes of a neurostimulator implanted in the subject, an amplitude-modulated high-frequency (AM-HF) sinusoidal current (I) according to:
$$I = {I}_{a} sin ( 2 π {f}_{c} t ) + {I}_{b} sin ( 2 π ( {f}_{c} + {f}_{b} ) t ) \qquad ( 1 )$$
wherein Iₐ and I_{b} are current values so that the maximum current amplitude is (Iₐ+I_{b}) and the minimum current amplitude value is ∥I_{b}−Iₐ∥, f_{c} is about 800 Hz to about 20 kHz, f_{b} is about 1 Hz to about 500 Hz, and t is time.

2. The method of claim 1, wherein the AM-HF sinusoidal current (I) is according to:
$$I = {I}_{p} sin ( 2 π {f}_{c} t ) + {I}_{p} sin ( 2 π ( {f}_{c} + {f}_{b} ) t ) \qquad ( 2 )$$
wherein 2*Iₚ is the maximum current amplitude, f_{c} is about 1 kHz to about 20 kHz, f_{b} is about 1 Hz to about 500 Hz, and t is time.

3. The method of claim 1, wherein the stimulation is applied to a peripheral nerve innervating a limb of the subject.

4. The method of claim 1, wherein the stimulation is provided to a sensory nerve innervating an amputated limb of the subject, and wherein the stimulation is provided in an amount effective to reduce phantom limb pain in the subject.

5. The method of claim 1, wherein the stimulation is provided to a sensory nerve innervating an amputated limb of the subject, and wherein the stimulation is provided in response to activation of a sensor detecting movement and/or position of the amputated limb or movement and/or position of a physical or virtual prosthesis of the amputated limb.

6. The method of claim 5, wherein the sensor is any one of a gyroscope, an electrogoniometer, a textile piezoresistive sensor, or a pressure sensor located in or on the limb or the prosthesis of the subject

7. The method of claim 5, wherein the sensor detects pressure at the fingertips of a prosthetic hand or in the sole of a prosthetic foot.

8. The method of claim 5, wherein the sensor detects a position of the limb, the stump of the limb, the prosthesis, or the virtual prosthesis.

9. The method of claim 8, wherein the virtual prosthesis of the limb is a computer-generated image of the limb and the movement of the computer-generated image of the limb is observed or controlled by the subject.

10. The method of claim 1, wherein the stimulation is provided to a peripheral nerve innervating a limb of the subject with complete or partial paralysis due to spinal cord injury or stroke.

11. The method of claim 10, wherein the stimulation is provided in an amount effective to cause reflexive muscle contraction in the limb of the subject with complete or partial paralysis due to spinal cord injury or stroke.

12. The method of claim 1, wherein the stimulation is applied to a peripheral nerve innervating a limb of the subject, and the method further comprises calibrating the stimulation to induce sensations of pressure or touch in the limb.

13. The method of claim 3, wherein the neurostimulator is implanted at the dorsal rootlets of sensory neurons innervating the limb of the subject.

14. The method of claim 3, wherein the neurostimulator is implanted at the lateral spinal cord adjacent to the dorsal rootlets of the sensory neurons innervating the limb of the subject.

15. The method of claim 3, wherein the neurostimulator is implanted on peripheral neurons innervating the limb of the subject.

16. The method of claim 1, wherein the electrodes of the neurostimulator are in the form of a nerve cuff, a microelectrode array, a deep brain stimulator, or a spinal probe.

17. The method of claim 1, wherein the neurostimulator is an external or implanted pulse generator.

18. The method of claim 1, further comprising implanting the neurostimulator in the subject.

19. A method for providing asynchronous and stochastic neural activity in a limb of a subject with an amputation of the limb, comprising:
providing a therapeutically effective amount of stimulation to peripheral neurons that innervate the limb of the subject and/or dorsal rootlets, or lateral spinal cord adjacent to the dorsal rootlets, of one or more sensory neurons that innervate the limb of the subject, wherein:
the stimulation is provided with one or more electrodes of a neurostimulator that are implanted at the peripheral neurons that innervate the limb of the subject and/or dorsal rootlets or the lateral spinal cord adjacent to the dorsal rootlets of the one or more sensory neurons innervating the limb of the subject;
the one or more electrodes are activated to provide the stimulation in response to activation of a sensor detecting movement and/or position of the limb or movement and/or position of a physical or virtual prosthesis of the limb; and
wherein the stimulation is amplitude-modulated high-frequency (AM-HF) sinusoidal current (I) according to:
$$I = {I}_{a} sin ( 2 π {f}_{c} t ) + {I}_{b} sin ( 2 π ( {f}_{c} + {f}_{b} ) t ) \qquad ( 1 )$$
wherein Iₐ and I_{b} are current values so that the maximum current amplitude is (Iₐ+I_{b}) and the minimum current amplitude value is ∥I_{b}−Iₐ∥, f_{c} is about 800 Hz to about 20 kHz, f_{b} is about 1 Hz to about 500 Hz, and t is time.

20. A method for control of a prosthetic limb of a subject, comprising:
providing a therapeutically effective amount of stimulation to peripheral neurons that innervate the limb of the subject and/or dorsal rootlets, or lateral spinal cord adjacent to the dorsal rootlets, of sensory neurons innervating an amputated limb of the subject, wherein the subject uses a prosthesis of the amputated limb; and wherein:
the stimulation is provided with one or more electrodes of a neurostimulator that are implanted at peripheral neurons that innervate the limb of the subject and/or the dorsal rootlets or the lateral spinal cord adjacent to the dorsal rootlets of the sensory neurons innervating the amputated limb;
the one or more electrodes are activated to provide the stimulation in response to activation of a sensor detecting movement and/or position of a stump of the limb or the prosthesis or in response to movement and/or position of a virtual prosthesis of the limb;
wherein the stimulation is amplitude-modulated high-frequency (AM-HF) sinusoidal current (I) according to:
$$I = {I}_{a} sin ( 2 π {f}_{c} t ) + {I}_{b} sin ( 2 π ( {f}_{c} + {f}_{b} ) t ) \qquad ( 1 )$$
wherein Iₐ and I_{b} are current values so that the maximum current amplitude is (Iₐ+I_{b}) and the minimum current amplitude value is ∥I_{b}−Iₐ∥, f_{c} is about 800 Hz to about 20 kHz, f_{b} is about 1 Hz to about 500 Hz, and t is time; and
and wherein the stimulation controls the prosthesis of the limb by the subject.
